(19) United States
(12) Patent Application Publication
(10) Pub. No.: US 2021/0154985 A1
(43) Pub. Date: May 27, 2021

(54) ANTIMICROBIAL ASSEMBLIES

(71) Applicant: CORNING INCORPORATED, Corning, NY (US)

(72) Inventors: Bavani Balakrisnan, Corning, NY (US); Brian Joseph Hanlon, Corning, NY (US); Joydeep Lahiri, Corning, NY (US); Bryan Patrick Livengood, Nicholasville, KY (US); Timothy James Orsley, San Jose, CA (US)

(21) Appl. No.: 17/091,844

(22) Filed: Nov. 6, 2020

Related U.S. Application Data
(60) Provisional application No. 62/938,396, filed on Nov. 21, 2019.

(51) Int. Cl. B32B 27/14; B32B 5/16

(52) U.S. Cl. CPC B32B 27/14; B82Y 30/00; B32B 5/16

(57) ABSTRACT

According to various examples of the present disclosure, an antimicrobial assembly includes a substrate comprising a matrix material having opposed first and second surfaces with a total thickness of the substrate defined therebetween. The assembly further includes an antimicrobial composition bound to the substrate and heterogeneously distributed about the antimicrobial assembly.

### CROSS REFERENCE TO RELATED APPLICATIONS

This application claims the benefit of priority under 35 U.S.C. § 119 of U.S. Provisional Application No. 62/938,396 filed Nov. 21, 2019, the content of which is incorporated herein by reference in its entirety.

### BACKGROUND

Various assemblies can be exposed to a number of undesired contaminants such as bacteria, viruses, mildew, mold, fungi, algae and the like. Exposure to these contaminants can render the assemblies visually unattractive or unsuitable for a particular purpose or present a health hazard. It can therefore be desirable to mitigate the ability of the undesired contaminants to thrive once in contact with a coating or composition.

### SUMMARY OF THE DISCLOSURE

According to various examples of the present disclosure, an antimicrobial assembly includes a substrate comprising a matrix material having opposed first and second surfaces with a total thickness of the substrate defined therebetween. The assembly further includes an antimicrobial composition bound to the substrate and heterogeneously distributed about the antimicrobial assembly.

According to various examples of the present disclosure, an antimicrobial assembly includes a substrate comprising a polymeric material, a ceramic, a metal, or a combination thereof and having opposed first and second surfaces with a thickness of the substrate defined therebetween. The assembly further includes an inorganic glass comprising copper particle bound to the substrate and heterogeneously distributed about the assembly.

According to various examples of the present disclosure, a method of forming the antimicrobial assembly is described. The antimicrobial assembly includes a substrate comprising a matrix material having opposed first and second surfaces with a total thickness of the substrate defined therebetween. The assembly further includes an antimicrobial composition bound to the substrate and heterogeneously distributed about the antimicrobial assembly. The method includes infusing the antimicrobial composition to the first surface of the substrate. The method further includes infusing the antimicrobial composition at least partially to the first surface of the substrate to achieve the heterogenous distribution of the antimicrobial composition.

### BRIEF DESCRIPTION OF THE FIGURES

The drawings illustrate generally, by way of example, but not by way of limitation, various embodiments discussed in the present document.

FIG. 1 is a cross-sectional schematic view of an antimicrobial assembly, in accordance with various examples.

### DETAILED DESCRIPTION

Reference will now be made in detail to certain examples of the disclosed subject matter. While the disclosed subject matter will be described in conjunction with the enumerated claims, it will be understood that the exemplified subject matter is not intended to limit the claims to the disclosed subject matter.

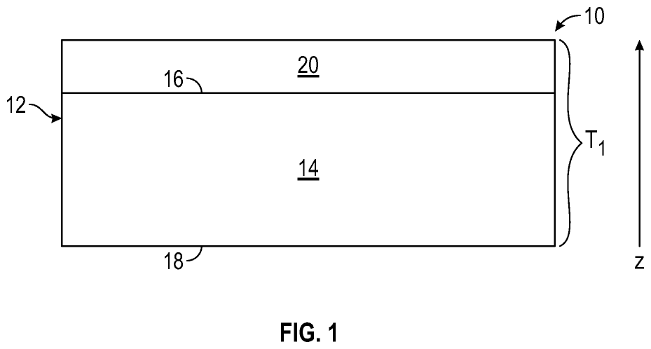

Various examples of the present disclosure relate to an antimicrobial assembly 10, as shown in FIG. 1, which is a cross-sectional schematic view of the antimicrobial assembly. The antimicrobial assemblies described herein can include a substrate 12. The substrate can include a matrix material 14 and has opposed first and second surfaces 16 and 18 with a total thickness Ti of the substrate defined therebetween. The antimicrobial assembly includes an antimicrobial composition 20. The antimicrobial composition is distributed about the antimicrobial assembly in a heterogenous manner. By “heterogenous” it is meant that the antimicrobial composition is not evenly distributed throughout the thickness of the substrate, but instead, is concentrated in certain regions of the substrate such that one region of the substrate may have a first quantity of the antimicrobial composition and a second region of the substrate may have less or even no antimicrobial composition present.

The antimicrobial assemblies described herein can take many different forms. For example, the antimicrobial assemblies can be chosen from many household and commercial products. As non-limiting examples, the assemblies can include keyboards, touch panel table tops, molded furniture, medical equipment, and the like. Given the vast scope of products to which the antimicrobial assembly can be incorporated, the profile or shape of the antimicrobial assembly can conform to any suitable profile or shape. For example the profile or shape of the antimicrobial assembly can be linear or non-linear. With respect to non-linear shapes, the antimicrobial assembly can have a curved, undulating, or even polygonal shape or profile. In some examples, the profile of the first surface can differ from the second surface. In some examples, the first surface and the second surface can merge to be one continuous surface and the heterogenous distribution of the antimicrobial composition can be achieved by having a portion of the antimicrobial composition distributed on the first and second surfaces with substantially no antimicrobial composition located in the interior of the antimicrobial assembly.

According to various examples, there are various non-limiting benefits to using the antimicrobial assemblies described herein. For example, having a heterogonous distribution of the antimicrobial composition can allow for the antimicrobial composition to be concentrated in areas where it is more useful. For example, if it is far more likely that the first surface of the substrate will come into contact with a microbe, as opposed to a second surface or an internal region of the substrate, the antimicrobial composition can be substantially located in a predetermined position and concentrated proximate to the first surface (relative to a similar assembly having a substantially homogenous distribution of the antimicrobial composition) to increase the overall antimicrobial activity of the antimicrobial assembly.

The matrix material of the antimicrobial assembly can include many suitable materials. For example, the matrix material can include a glass, a polymeric material, a ceramic, a metal, or a combination thereof. Examples of glass materials can include soda-lime glass, borosilicate glass, alumino-silicate glass, alkali-borosilicate glass, aluminoborosilcate glass, an alkalialuminosilicate glass, or a mixture thereof. Examples of suitable polymeric materials can include a thermoplastic polymeric material. Further examples of suitable polymeric materials can include a polycarbonate, a polyolefin, a polystyrene, a polyacrylate, a polyamide, a polyvinyl acetate, a polyurethane, a polybenzimidazole, a polyimide, a polyester, a polyetherimine, a fluoropolymer, a polyvinyl chloride, a polyoxymethylene, a polylactic acid, an acrylonitrile butadiene, a polyether ether ketone, a polyphenylene sulfide, styrene polymer, copolymers thereof, or mixtures thereof. Examples of suitable ceramics can include a fused aluminum oxide, a heat-treated aluminum oxide, a ceramic aluminum oxide, a sintered aluminum oxide, a silicon carbide material, a titanium diboride, a boron carbide, a tungsten carbide, a titanium carbide, a diamond, a cubic boron nitride, a garnet, a fused alumina-zirconia, a cerium oxide, a zirconium oxide, a titanium oxide or a mixture thereof. Examples of suitable metals can include a stainless steel, aluminum, tin, titanium, alloys thereof, and mixtures thereof.

According to various examples, the antimicrobial composition can be understood to be an additive that can be incorporated into the substrate to add biocidal activity thereto. The antimicrobial composition can be characterized by the type of biocidal activity it provides. For example, the antimicrobial composition can be understood to be an antibacterial composition, an antifungal composition, an antiviral composition, an antiparasitic composition, or a combination thereof (e.g., the composition possess a plurality of biocidal activities).

The antimicrobial compositions described herein can include suitable constituents for providing the antimicrobial activities. For example, the antimicrobial compositions can include elemental copper, copper ion, elemental silver, silver ion, zinc, acrylates, phosphate, quaternary ammonium compounds, or a mixture thereof. In some examples the antimicrobial compositions can include one or more inorganic glass comprising copper particles. A median size of the one or more inorganic glass comprising copper particles can be in a range of from about 0.5 nm to about 15 μm, about 3 μm to about 8 μm, about 4 μm to about 6 μm, less than, equal to, or greater than about 0.5 nm, 1 nm, 10 nm, 50 nm, 100 nm, 200 nm, 300 nm, 400 nm, 500 nm, 600 nm, 700 nm, 800 nm, 900 nm, 1 μm, 2 μm, 3 μm, 4 μm, 5 μm, 6 μm, 7 μm, 8 μm, 9 μm, 10 μm, 11 μm, 12 μm, 13 μm, 14 μm, or about 15 μm. The median size can be determined by analyzing the major dimension of the individual inorganic glass comprising copper particles. The major dimension, on an individual basis, can be a measurement of the diameter, width, or length of the individual inorganic glass comprising copper particles.

The one or more inorganic glass comprising copper particles can be present in a range of from about 3 wt % to about 88 wt % of the antimicrobial composition, about 10 wt % to about 87 wt %, about 42 wt % to about 85 wt %, less than, equal to, or greater than about 3 wt %, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, or about 88 wt %. The inorganic glass portion of the individual inorganic glass comprising copper particle component can include any suitable material such as SiO₂, Al₂O₃, CaO, MgO, P₂O₅, B₂O₃, K₂O, ZnO, Fe₂O₃, nanoparticles thereof, or a mixture thereof.

The copper of the inorganic glass comprising copper particles can be present in an individual inorganic glass comprising copper particle in any suitable amount. For example, the copper can be present in a range of from about 5 wt % to about 80 wt % of the individual inorganic glass comprising copper particle, about 10 wt % to about 70 wt %, about 25 wt % to about 35 wt %, about 40 wt % to about 60 wt %, about 45 wt % to about 55 wt %, less than, equal to, or greater than about 5 wt %, 10, 15, 20, 25, 30, 35, 40, 45, 50, 55, 60, 65, 70, 75, or about 80 wt %. In each inorganic glass comprising copper particle, the copper portion can independently include a Cu metal, Cu⁺, Cu²⁺, or a combination of Cu⁺ and Cu²⁺. The copper can be non-complexed or can have a ligand bonded thereto to form a complex. According to some examples, the Cu⁺ can be present as cuprous oxide (Cu₂O). In some examples the cuprous oxide particles can be nanometer sized (e.g., between 0.5 nm and 999 nm) and encased in any of the glasses described herein.

Although the inorganic glass comprising copper particle is effective as a biocidal agent, a potential drawback is that the copper offers numerous opportunities for ligands to attach thereto, resulting in complexes that can alter the color of the antimicrobial assembly to which it is ultimately incorporated. However, it is possible to pair the inorganic glass comprising copper particles with various additional additives in order to limit the extent to which the copper is complexed and therefore the color of the antimicrobial composition is altered from a standard. According to some examples, however, the color induced by the antimicrobial composition can be useful in creating a contrast between the composition and the matrix material to allow an observer to visually confirm the presence of the antimicrobial composition. The color of the antimicrobial composition can also be leveraged as a decoration in the antimicrobial assembly. This can also help to allow a person to properly align the assembly such that it is properly positioned to increase contact between the antimicrobial composition and any microbes. The contrast in color can also be used to create visual patterns for aesthetic purposes in the assembly.

In examples where a contrast in color is not desired, in an antimicrobial assembly to which the antimicrobial composition is incorporated, it is possible to achieve a CIEL*a*b* delta E* between the observed color and a standard (e.g., a dispersion or antimicrobial composition including the same constituents, but are free of or include a different amount of the glass comprising copper) of less than about 15, less than about 14, less than about 13, less than about 12, less than about 11, less than about 10, less than about 9, less than about 8, less than about 7, less than about 6, less than about 5, less than about 4, less than about 3, less than about 2, less than about 1, in a range of from about 1 to about 15, about 2 to about 13, about 5 to about 10, about 3 to about 8, about 4 to about 7, about 5 to about 6, less than, equal to, or greater than about 1, 2, 3, 4, 5, 6, 7, 8, 9, 10, 11, 12, 13, 14, 15, 16, 17, 18, 19, 20, 21, 22, 23, 24, or about 25. As understood, the CIEL*a*b* color scale is a scale for determining a color. Using this test, the difference (e.g., a delta E*) in color between a standard and observed color can be measured. In this manner, the extent to which the desired color of a composition is altered by the components therein can be measured.

In operation, the copper from the antimicrobial composition can be released into the antimicrobial assembly to interact with and kill unwanted biological contaminants such as microbes to which the antimicrobial assembly is exposed. Examples of microbes that the copper can kill include Staphylococcus aureus, Enterobacter aerogenes, Pseudomomas aeruginosa, Methicillin Resistant, E. coli, Enterobacter cloacae, Acinetobacter baumannii, Enterococcus faecalis, Klebsiella pneumoniae, Klebsiella aerogenes, Staphylococcus aureus, and mixtures thereof. Examples of viruses that the copper can kill include Influenza H1N1, Adenovirus 5, and Norovirus. An example of a fungi the copper can kill includes Candida auris. The effectiveness of the antimicrobial composition as a biocidal agent can be measured as a function of the antimicrobial composition's log reduction. The antimicrobial composition's log reduction value can be relevant to its ability to kill biological organisms to which it is exposed.

According to various examples, a log reduction of the antimicrobial composition, or of the assembly to which it is incorporated, can be at least about 2, at least about 3, at least about 4, at least about 5, at least about 6, at least about 7, at least about 8, at least about 9, at least about 10, in a range of from about 1 to about 10, about 3 to about 7, about 4 to about 6, or less than, equal to, or greater than about 1, 2, 3, 4, 5, 6, 7, 8, 9, or about 10. The log reduction value can be measured according to the EPA's draft protocol on bactericidal efficacy of copper-containing surface products, 2016 or the JIS Z 2801 Test for Antimicrobial Activity of Plastics. An example of an advantage to using the inorganic glass comprising copper components described herein is that the copper is less corrosive, and toxic, than many organic biocidal compounds that are included in other applications.

The antimicrobial compositions described herein can be formed according to any suitable method. For example, the antimicrobial compositions described herein can be formed by combining any combination or sub-combination of the components described herein, of the antimicrobial composition. The antimicrobial composition can then be mixed at a low or high shear in an aqueous medium. In further examples, all of the components of the antimicrobial composition, can be present as a powder mixture. The powder mixture can be mixed and then water or an organic solvent can be added to disperse the components and form a liquid antimicrobial composition.

After the antimicrobial composition is formed, it can be contacted with the substrate to form the antimicrobial assembly. Upon contact, the antimicrobial composition is at least partially infused into the substrate. Infusion leads to the antimicrobial composition being bound or integral to the first surface of the antimicrobial assembly. According to various examples, the antimicrobial composition, or at least a portion thereof is exposed on the first surface. According to some examples, infusion can lead to substantially no antimicrobial composition being exposed on an external surface of the substrate. Alternatively, infusion can be considered to be partial in that some portion of the antimicrobial composition is embedded in the substrate while another portion of the antimicrobial composition is exposed on an external surface of the substrate.

Within the scope of this disclosure, infusion can include various methods or procedures. For example, infusion can include softening the substrate. With specific reference to examples where the matrix material includes a thermoplastic polymer or mixture of thermoplastic polymers, the substrate can be heated to, or in excess of, a first glass transition temperature of the thermoplastic polymer. In examples where the thermoplastic polymer includes multiple glass transition temperatures, the relevant thermoplastic polymer can be the lowest glass transition temperature. The substrate can be heated for a predetermined amount of time to control the degree to which the substrate is ultimately heated. Controlling the degree to which the substrate is heated can be useful to ensure that a first portion of the substrate adjacent to the first surface of the substrate is heated to a higher temperature than a second portion of the substrate distal to the first surface of the substrate. Controlling the degree to which the substrate is heated can create a heat gradient in the substrate. For example, the temperature at or proximate to the first surface can be greater than the temperature at or proximate to the second surface with the temperature decreasing along the z-direction therebetween. This can help to ensure that the antimicrobial composition is largely infused substantially in the heated or softened region of the substrate, thus producing the heterogenous distribution of the antimicrobial composition.

The antimicrobial composition can be delivered to the heated surface of the substrate in a variety of ways. For example, the antimicrobial composition can be sprayed onto the substrate. In examples where the antimicrobial composition is sprayed, the antimicrobial composition can be dispersed in a solvent, such as a heat labile solvent. Examples of heat labile solvents include water or an organic solvent. Examples of suitable organic solvents can include isopropanol, xylene, butyl acetate, or a mixture thereof. Where the solvent is a heat labile solvent, it can be desirable to heat the solvent to aid in softening the substrate upon infusion.

In still further examples, the antimicrobial composition can be delivered to the substrate when the substrate is in an uncured or partially cured state. In this manner, the antimicrobial composition is allowed to bind to the substrate while at least a portion of the substrate is in a softened state, thus allowing for infusion of the of the antimicrobial composition into the substrate. Following infusion, the substrate can be cooled to re-solidify, or the substrate can be fully cured (e.g., through exposure to ultra violet radiation) to form the final antimicrobial assembly. In some examples, the substrate, whether cured or partially cured can be deposited in a mold. The antimicrobial composition can then be contacted with the substrate mold and the mold can be heated to aid in infusion. To further aid in infusion, the mold can be pressed to force contact between the antimicrobial composition and the substrate. In some examples, where the substrate includes a ceramic material, infusion can occur while the substrate is in a pre-fired “green state”. Following infusion, the substrate can be fired to yield the final ceramic. In some examples, to mitigate the potential degradation of the antimicrobial composition, firing can be done in an inert atmosphere

Some of the infusion techniques can be further enhanced by altering the first surface prior to infusion. For example, the first surface can be chemically softened or plasticized to allow infiltration of the antimicrobial composition. In additional examples, the first surface can be etched or include a pore-inducing agent to facilitate infusion of the antimicrobial composition thereto.

In some examples, the antimicrobial assembly can be manufactured according to an additive manufacturing process. Additive manufacturing (AM) fabricates a product directly from a model such as a Computer Aided Design (CAD) model regardless of geometric complexity. Examples of additive manufacturing processes include extrusion freeform fabrication (EFF), Stereolithography (SLA), binder/ink jetting, and Selective Laser Sintering (SLS). Generally, an SLA process involves filling a tank with photosensitive resin, which solidifies once exposed to UV light. A dynamic digital image (or a laser dot) can then be projected onto the liquid surface to solidify the resin layer by layer. Generally, SLS uses a laser as the power source to sinter powdered material. The laser is aimed at points in space defined by the CAD model. The laser causes the material to sinter and bind material together to create a solid structure. Generally, EFF uses a continuous filament of a thermoplastic material. The filament can be fed from a large coil through a moving, heated printer extruder head, and is deposited on the growing work. The print head can be moved under computer control to define the printed shape. The head can be moved in two dimensions to deposit one horizontal plane, or layer, at a time. To create a vertical layer the work or the print head can then be moved vertically by a small amount to begin a new layer. The speed of the extruder head may also be controlled to stop and start deposition and form an interrupted plane without stringing or dribbling between sections.

In any additive manufacturing technique, the heterogenous distribution of the antimicrobial composition can be achieved by controlling the components in each layer that is deposited. For example, a first plurality of layers can include the substrate matrix material and be substantially free of the antimicrobial composition. Conversely, another deposited layer or second plurality of layers can include the antimicrobial composition. The antimicrobial composition can be included in the second layer of plurality of layers by extruding the layer with the antimicrobial composition. Thus, the content and location of any layer or layers that include the antimicrobial composition in the antimicrobial assembly can be controlled through additive manufacturing. The location of the antimicrobial composition in each layer can also be controlled such that the antimicrobial composition is arranged as a pattern or only disposed over a portion of the total area of a layer. For example, the antimicrobial composition can be disposed over 5% to about 100% of a total surface area of a layer, about 10% to about 90%, about 20% to about 80%, about 30%, to about 70%, about 40% to about 60%, less than, equal to, or greater than about 5%, 10, 15, 20, 25, 30, 35, 40, 45, 50, 55, 60, 65, 70, 75, 80, 85, 90, 95, or about 100%. The pattern of the antimicrobial composition can be continuous or discontinuous. Patterning of the antimicrobial composition can be accomplished in a manner substantially similar to that of patterning color in a full color 3D printer.

In the final antimicrobial assembly, the heterogenous distribution of the antimicrobial composition can ensure that a major portion of the antimicrobial composition is located proximate to a surface of the substrate. For example, in the substrate, over 50 wt % of the antimicrobial composition can be found between a plane defined by a surface of the dried product and a substantially parallel plane extending through the center of the substrate. For example, about 50 wt % to about 100 wt % of the antimicrobial composition can be located proximate to the surface of the substrate, or about 55 wt % to about 95 wt %, about 60 wt % to about 90 wt %, about 65 wt % to about 85 wt %, about 70 wt % to about 80 wt %, less than, equal to, or greater than about 55 wt %, 60, 65, 70, 75, 80, 85, 90, 95, or about 100 wt %. With respect to the depth to which the antimicrobial composition can penetrate the substrate, the antimicrobial composition can be distributed about the antimicrobial assembly through about 5%, 10%, 15%, 20%, 25%, 30%, 35%, 40%, 45%, or about 50% of the total thickness of the antimicrobial assembly measured from one of the first or second surface of the substrate to a point between the the first or second surface of the substrate and the other of the first or second surface of the substrate.

The terms and expressions that have been employed are used as terms of description and not of limitation, and there is no intention in the use of such terms and expressions of excluding any equivalents of the features shown and described or portions thereof, but it is recognized that various modifications are possible within the scope of the examples of the present disclosure. Thus, it should be understood that although the present disclosure has been specifically disclosed by specific examples and optional features, modification and variation of the concepts herein disclosed can be resorted to by those of ordinary skill in the art, and that such modifications and variations are considered to be within the scope of examples of the present disclosure.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading can occur within or outside of that particular section.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

### ADDITIONAL EXAMPLES

The following exemplary examples are provided, the numbering of which is not to be construed as designating levels of importance:

Example 1 provides an antimicrobial assembly comprising:

a substrate comprising a matrix material having opposed first and second surfaces with a total thickness of the substrate defined therebetween; and

an antimicrobial composition bound to the substrate and heterogeneously distributed about the antimicrobial assembly.

Example 2 provides the antimicrobial assembly of Example 1, wherein the matrix material comprises a polymeric material, a ceramic, a metal, or a mixture thereof.

Example 3 provides the antimicrobial assembly of Example 2, wherein the polymeric material comprises a thermoplastic polymeric material.

Example 4 provides the antimicrobial assembly of any one of Examples 2 or 3, wherein the polymeric material comprises a polycarbonate, a polyolefin, a polystyrene, a polyacrylate, a polyamide, a polyvinyl acetate, a polyurethane, a polybenzimidazole, a polyimide, a polyester, a polyetherimine, a fluoropolymer, a polyvinyl chloride, a polyoxymethylene, a polylactic acid, a acrylonitrile butadiene, a polyether ether ketone, a polyphenylene sulfide, styrene polymer, copolymers thereof, or mixtures thereof.

Example 5 provides the antimicrobial assembly of any one of Examples 2-4, wherein the ceramic comprises a fused aluminum oxide, a heat-treated aluminum oxide, a ceramic aluminum oxide, a sintered aluminum oxide, a silicon carbide material, a titanium diboride, a boron carbide, a tungsten carbide, a titanium carbide, a diamond, a cubic boron nitride, a garnet, a fused alumina-zirconia, a cerium oxide, a zirconium oxide, a titanium oxide or a mixture thereof.

Example 6 provides the antimicrobial assembly of any one of Examples 2-5, the metal comprises a stainless steel, aluminum, tin, titanium, alloys thereof, and mixtures thereof.

Example 7 provides the antimicrobial assembly of any one of Examples 1-6, wherein the antimicrobial composition comprises an antibacterial, an antifungal, an antiviral, an antiparasitic, or a combination thereof.

Example 8 provides the antimicrobial assembly of any one of Examples 1-7, wherein the antimicrobial composition comprises elemental copper, copper ion, elemental silver, silver ion, zinc, acrylates, phosphate, quaternary ammonium compounds or a mixture thereof.

Example 9 provides the antimicrobial assembly of any one of Examples 1-8, wherein the antimicrobial composition includes an inorganic glass comprising copper particle.

Example 10 provides the antimicrobial assembly of any one Example 9, wherein the inorganic glass comprising copper particle are in a range of from about 10 wt % to about 90 wt % of the antimicrobial composition.

Example 11 provides the antimicrobial assembly of any one of Examples 9 or 10 the inorganic glass comprising copper particle is in a range of from about 10 wt % to about 22 wt % of the antimicrobial composition.

Example 12 provides the antimicrobial assembly of any one of Examples 9-11, wherein a median size of the inorganic glass comprising copper particle is in a range of from about 0.5 nm to about 15 μm.

Example 13 provides the antimicrobial assembly of any one of Examples 9-12, wherein the inorganic glass comprising copper particle comprises an inorganic glass comprising SiO₂, Al₂O₃, CaO, MgO, P₂O₅, B₂O₃, K₂O, ZnO, Fe₂O₃, or a mixture thereof.

Example 14 provides the antimicrobial assembly of any one of Examples 9-13, wherein the inorganic glass comprising copper particle comprises an inorganic glass comprising SiO₂ nanoparticles, alumina nanoparticles, or mixtures thereof.

Example 15 provides the antimicrobial assembly of any one of Examples 9-14, wherein the copper is independently in a range of from about 25 wt % to about 40 wt % of the inorganic glass comprising copper particle.

Example 16 provides the antimicrobial assembly of any one of Examples 9-15, wherein the copper is independently Cu metal, Cu⁺, Cu²⁺, or a mixture of Cu⁺ and Cu²⁺.

Example 17 provides the antimicrobial assembly of any one of Examples 9-16, wherein the inorganic glass comprising copper is functionalized with a silane.

Example 18 provides the antimicrobial assembly of any one of Examples 9-17, wherein the copper comprises Cu₂O.

Example 19 provides the antimicrobial assembly of any one of Examples 1-18, wherein the antimicrobial composition further comprises at least one colorant.

Example 20 provides the antimicrobial assembly of Example 19, wherein the colorant is in a range of from about 0.1 wt % to about 22 wt % of the antimicrobial composition.

Example 21 provides the antimicrobial assembly of any one of Examples 19 or 20, wherein the colorant is in a range of from about 1 wt % to about 5 wt % of the antimicrobial composition.

Example 22 provides the antimicrobial assembly of any one of Examples 19-21, further comprising at least one extender.

Example 23 provides the antimicrobial assembly of Example 22, wherein the extender is in a range of from about 0.1 wt % to about 15 wt % of the antimicrobial composition.

Example 24 provides the antimicrobial assembly of any one of Examples 22 or 23, wherein the extender is in a range of from about 1 wt % to about 5 wt % of the antimicrobial composition.

Example 25 provides the antimicrobial assembly of any one of Examples 19-24, wherein the colorant or extender comprises clay, talc, TiO₂, aluminum trihydrate, nepheline syenite, CaCO₃, silica, a flattening agent, barium sulfate, zinc oxide, or a mixture thereof.

Example 26 provides the antimicrobial assembly of any one of Examples 19-25, further comprising at least one pigment in the antimicrobial composition.

Example 27 provides the antimicrobial assembly of Example 26, wherein the pigment is in a range of from about 0.1 wt % to about 30 wt % of the antimicrobial composition.

Example 28 provides the antimicrobial assembly of any one of Examples 26 or 27, wherein the pigment is in a range of from about 1 wt % to about 5 wt % of the antimicrobial composition.

Example 29 provides the antimicrobial assembly of any one of Examples 26-28, wherein the pigment is TiO₂.

Example 30 provides the antimicrobial assembly of any one of Examples 1-29, wherein the antimicrobial composition is at least partially dispersed on the first surface of the substrate.

Example 31 provides the antimicrobial assembly of any one of Examples 1-30, wherein the antimicrobial composition is at least partially dispersed within the substrate.

Example 32 provides the antimicrobial assembly of any one of Examples 1-31, wherein the antimicrobial composition is fully dispersed within the substrate.

Example 33 provides the antimicrobial assembly of any one of Examples 1-32, wherein the antimicrobial composition is distributed about the antimicrobial assembly through about 50% of the total thickness of the antimicrobial assembly measured from the first surface of the substrate to a point between the first surface of the substrate and the second surface the first surface of the substrate.

Example 34 provides the antimicrobial assembly of any one of Examples 1-33, wherein the antimicrobial composition is distributed about the antimicrobial assembly through about 25% of the total thickness of the antimicrobial assembly measured from the first surface of the substrate to a point between the first surface of the substrate and the second surface the first surface of the substrate.

Example 35 provides the antimicrobial assembly of any one of Examples 1-34, wherein the antimicrobial composition is distributed about the antimicrobial assembly through about 10% of the total thickness of the antimicrobial assembly measured from the first surface of the substrate to a point between the first surface of the substrate and the second surface the first surface of the substrate.

Example 36 provides the antimicrobial assembly of any one of Examples 1-35, wherein the antimicrobial composition is distributed about the antimicrobial assembly through about 5% of the total thickness of the antimicrobial assembly measured from the first surface of the substrate to a point between the first surface of the substrate and the second surface the first surface of the substrate.

Example 37 provides an antimicrobial assembly comprising:

a substrate comprising a polymeric material, a ceramic, a metal, or a combination thereof and having opposed first and second surfaces with a thickness of the substrate defined therebetween; and

an inorganic glass comprising copper particle bound to the substrate and heterogeneously distributed about the assembly.

Example 38 provides a method of forming the antimicrobial assembly of any one of Examples 1-37, the method comprising:

contacting the antimicrobial composition with the first surface of the substrate; and

infusing the antimicrobial composition at least partially to the first surface of the substrate to achieve the heterogenous distribution of the antimicrobial composition.

Example 39 provides the method of Example 38, wherein infusing comprises heating the substrate above a glass transition temperature of the matrix material.

Example 40 provides the method of Example 39, wherein the antimicrobial composition is contacted with the first surface of the substrate for a predetermined amount of time following heating of the substrate.

Example 41 provides the method of any one of Examples 39 or 40, wherein a first portion of the substrate adjacent to the first surface of the substrate is heated to a higher temperature than a second portion of the substrate distal to the first surface of the substrate.

Example 42 provides the method of any one of Examples 38-41, wherein infusing comprises spraying the antimicrobial composition to bind to the first surface of the substrate.

Example 43 provides the method of Example 42, wherein the antimicrobial composition comprises a solvent.

Example 44 provides the method of Example 43, wherein the solvent is heat labile.

Example 45 provides the method of any one of Examples 43 or 44, wherein the solvent comprises water or an organic material.

Example 46 provides the method of any one of Examples 43-45, wherein the organic material comprises isopropanol, xylene, butyl acetate, or a mixture thereof.

Example 47 provides the method of any one of Examples 43 or 46, further comprising heating the solvent.

Example 48 provides the method of any one of clams 38-47, wherein the substrate is at least partially cured or in a green state.

Example 49 provides the method of any one of Examples 38-48, wherein infusing comprises:

contacting the substrate or a precursor thereof with a mold;

contacting the first surface of the substrate with a suspension comprising the antimicrobial composition; and

pressing the substrate.

Example 50 provides the method of Example 49, further comprising heating the mold.

Example 51 provides the method of any one of Examples 38-50, further comprising manufacturing the substrate by additive manufacturing, wherein the antimicrobial composition is contacted with the substrate during additive manufacturing.

Example 52 provides the method of Example 51, wherein the antimicrobial composition is present in a layer of a substrate precursor material that is deposited during additive manufacturing.

Example 53 provides the method of any one of Examples 51 or 52, wherein the additive manufacturing process comprises selective laser sintering.

Example 54 provides the method of Example 53, wherein the assembly formed by additive manufacturing comprises plurality of layers and the antimicrobial composition is present in a subset of the total number of layers.

Example 55 provides the method of any one of Examples 53 or 54, wherein the antimicrobial composition is present in one layer of the plurality of layers.

Example 56 provides the method of any one of Examples 38-55, wherein the antimicrobial composition is coextruded with the matrix material.

Example 57 provides the method of any one of Examples 52-56, wherein the antimicrobial composition forms a pattern in the layer.

What is claimed is:

1. An antimicrobial assembly comprising:
a substrate comprising a matrix material having opposed first and second surfaces with a total thickness of the substrate defined therebetween; and
an antimicrobial composition infused into the first surface of the substrate and heterogeneously distributed about the antimicrobial assembly.

2. The antimicrobial assembly of claim 1, wherein the matrix material comprises a thermoplastic polymeric material.

3. The antimicrobial assembly of claim 2, wherein the polymeric material comprises a polycarbonate, a polyolefin, a polystyrene, a polyacrylate, a polyamide, a polyvinyl acetate, a polyurethane, a polybenzimidazole, a polyimide, a polyester, a polyetherimine, a fluoropolymer, a polyvinyl chloride, a polyoxymethylene, a polylactic acid, a acrylonitrile butadiene, a polyether ether ketone, a polyphenylene sulfide, styrene polymer, copolymers thereof, or mixtures thereof.

4. The antimicrobial assembly of claim 1, wherein the antimicrobial composition comprises an antibacterial, an antifungal, an antiviral, an antiparasitic, or a combination thereof.

5. The antimicrobial assembly of claim 1, wherein the antimicrobial composition comprises elemental copper, copper ion, elemental silver, silver ion, zinc, acrylates, phosphate, quaternary ammonium compounds or a mixture thereof.

6. The antimicrobial assembly of claim 1, wherein the antimicrobial composition comprises an inorganic glass comprising copper particle.

7. The antimicrobial assembly of claim 6, wherein the inorganic glass comprising copper particle is in a range of from about 10 wt % to about 90 wt % of the antimicrobial composition.

8. The antimicrobial assembly of claim 6, wherein the inorganic glass comprising copper particle comprises an inorganic glass comprising SiO₂, Al₂O₃, CaO, MgO, P₂O₅, B₂O₃, K₂O, ZnO, Fe₂O₃, or a mixture thereof.

9. The antimicrobial assembly of claim 6, wherein the inorganic glass comprising copper particle comprises an inorganic glass comprising SiO₂ nanoparticles, alumina nanoparticles, or mixtures thereof.

10. The antimicrobial assembly of claim 6, wherein the copper is independently Cu metal, Cu⁺, Cu²⁺, or a mixture of Cu⁺ and Cu²⁺.

11. The antimicrobial assembly of claim 1, wherein the antimicrobial composition is distributed about the antimicrobial assembly through about 50% of the total thickness of the antimicrobial assembly measured from the first surface of the substrate to a point between the first surface of the substrate and the second surface the first surface of the substrate.

12. The antimicrobial assembly of claim 1, wherein the antimicrobial composition is distributed about the antimicrobial assembly through about 25% of the total thickness of the antimicrobial assembly measured from the first surface of the substrate to a point between the first surface of the substrate and the second surface the first surface of the substrate.

13. A method of forming the antimicrobial assembly of claim 1, the method comprising:
contacting the antimicrobial composition with the first surface of the substrate; and
infusing the antimicrobial composition at least partially into the first surface of the substrate to achieve the heterogenous distribution of the antimicrobial composition.

14. The method of claim 13, wherein the infusing comprises heating the substrate above a glass transition temperature of the matrix material.

15. The method of claim 13, wherein the heating comprises heating a first portion of the substrate adjacent to the first surface of the substrate to a higher temperature than a second portion of the substrate distal to the first surface of the substrate.

16. The method of claim 13, wherein the infusing comprises spraying the antimicrobial composition to bind to the first surface of the substrate.

17. The method of claim 16, wherein:
the antimicrobial composition comprises a heat labile solvent; and
the infusing further comprises heating the antimicrobial composition to aid in softening the substrate during the infusion.

18. The method of claim 13, wherein:
during the contacting, the substrate is uncured or partially cured; and
the method comprises further curing the substrate following the infusion.

19. The method of claim 13, wherein the infusing comprises:
contacting the substrate or a precursor thereof with a mold;
contacting the first surface of the substrate with a suspension comprising the antimicrobial composition; and
pressing the substrate.

20. The method of claim 13, further comprising manufacturing the substrate by additive manufacturing, wherein
the antimicrobial composition is contacted with the substrate during additive manufacturing.
